(19) United States
(12) Patent Application Publication
(10) Pub. No.: US 2025/0042650 A1
(43) Pub. Date: Feb. 6, 2025

(54) Diaper Changing and Disposal Bag

(71) Applicant: MKT Ventures LLC, Phoenix, AZ (US)

(72) Inventor: Kristin Ann Tanner, Phoenix, AZ (US)

(21) Appl. No.: 18/795,010

(22) Filed: Aug. 5, 2024

Related U.S. Application Data
(63) Continuation-in-part of application No. 29/913,518, filed on Oct. 3, 2023.
(60) Provisional application No. 63/517,845, filed on Aug. 4, 2023.

(51) Int. Cl. A61F 13/551

(52) U.S. Cl. CPC B65F 1/0006; A47D 5/006; A61F 13/5512; B65F 2210/167; B65F 2240/132

(57) ABSTRACT

A foldable bag designed to provide a cost-effective, convenient, and sanitary way to dispose of a dirty diaper. The foldable bag comprises two pockets: a front pocket and a rear pocket. The front pocket receives the dirty diaper, while the rear pocket receives the front pocket. Once the diaper is secured in the front pocket, the user twists the front pocket to lock the odors within. Next, the user rolls up the front pocket until the front pocket reaches the top edge. Lastly, the user tucks the front pocket from the front side into the rear pocket on the rear side. Once tucked in place, the foldable bag can be properly discarded without emitting odors into the surrounding area. In a preferred embodiment, the upper section of the foldable bag folds outward to form a large surface area that can be used for changing a baby's diaper.

### FIELD OF THE INVENTION

The present invention relates generally to a diaper changing and disposal bag. More specifically, the present invention is a device that is designed with plastic and twists to lock in unpleasant odors.

### BACKGROUND OF THE INVENTION

Diaper disposal is a process of safely and hygienically disposing of used diapers, typically worn by infants or toddlers. It is an essential aspect of childcare, ensuring a clean and odor-free environment for both the baby and caregivers. Various methods and products are available for diaper disposal, including diaper pails, diaper genie systems, and disposable diaper bags. These systems are designed to seal in odors and prevent contamination, making the process more convenient and sanitary. Proper diaper disposal is crucial for maintaining cleanliness and preventing the spread of germs, contributing to a healthier and more comfortable environment for babies and those caring for them. Unfortunately, many current systems do not properly block odor or provide a sanitary surface when changing a diaper.

An objective of the present invention is to provide users with a diaper changing and disposal bag featuring a front pocket to place the waste in with an option to expand the disposal bag to create a barrier along a changing table. The present invention intends to provide users with a device that can be utilized for baby diapers as well as adult diapers. In order to accomplish that, a preferred embodiment of the present invention comprises a front side and a rear side. Thus, the present invention is a foldable bag that receives a dirty diaper, locking in germs and odor, twisting, and rolling up into a compact profile ready for disposal.

### SUMMARY OF THE INVENTION

The present invention is a disposable diaper bag to help with securing a diaper to limit the surrounding odor. The present invention seeks to provide users with a device that can roll up a diaper, wrapping the diaper in several layers of protective plastic. In order to accomplish this objective, the present invention comprises a front side where the diaper is initially rolled up. Further, the rear side is where the diaper is tucked in to create a small and secure profile. Thus, the present invention is a foldable bag that receives a dirty diaper, locking in germs and odor, twisting, and rolling up into a compact profile ready for disposal.

### BRIEF DESCRIPTION OF THE DRAWINGS

FIG. 1 is a top front left perspective view of the present invention.

FIG. 2 is a bottom rear right perspective view of the present invention.

FIG. 3 is a front elevational view of the present invention.

FIG. 4 is a rear elevational view of the present invention.

FIG. 5 is a right-side elevational view of the present invention.

FIG. 6 is a top front left perspective view of the present invention, shown in a folded configuration.

FIG. 7 is a bottom rear right perspective view of the present invention, shown in a folded configuration.

### DETAIL DESCRIPTIONS OF THE INVENTION

All illustrations of the drawings are for the purpose of describing selected versions of the present invention and are not intended to limit the scope of the present invention.

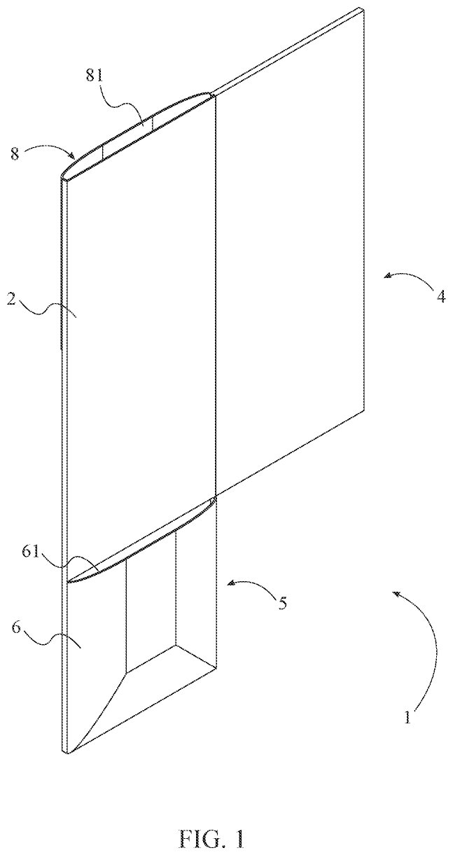

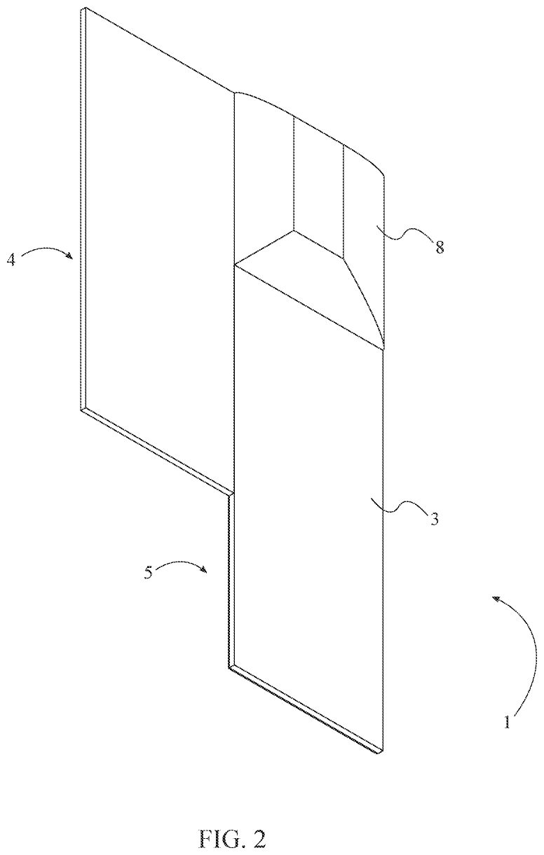

In reference to FIGS. 1-7, the present invention is diaper disposal and changing bag. An objective of the present invention is to provide users with a cost-effective, convenient, sanitary, odor trapping diaper disposal device. The present invention intends to provide the user with a specially designed foldable bag having two separate pockets. The front pocket 6 receives the dirty diaper, while the rear pocket 8 receives the front pocket 6 containing the dirty diaper. Thus, the foldable bag 1 is designed to lock in odors and prepare the dirty diaper for proper disposal. Preferably, the present invention is made from a plastic material designed to be disposable. Alternatively, the plastic material can be recyclable.

To use the foldable bag during diaper changing, the user first unfolds the upper section 4 along the front side 2 of the bag, which effectively doubles the width of the upper section. Once unfolded, the upper section 4 forms a large barrier surface for placing the baby on top of when changing the baby's diaper. After the diaper is changed, the user places the dirty diaper in the front pocket 6 and folds the upper section 4 back in place to secure germs and debris. If the foldable bag is not being used while changing the diaper, the upper section 4 can remain in the folded position, and the user simply places the dirty diaper in the front pocket 6.

After securing the dirty diaper in the front pocket 6, the user then twists the front pocket to lock the odors within. Next, the user rolls up the front pocket 6 along the front side 2 until reaching the top edge 80, thereby the disposed sealed bag is now wrapped further in a double layer of plastic. Lastly, the user tucks the front pocket 6 into the rear pocket 8 of the rear side 3. Once tucked in place, the foldable bag 1 can be properly discarded without letting odors or germs seep into the surrounding area.

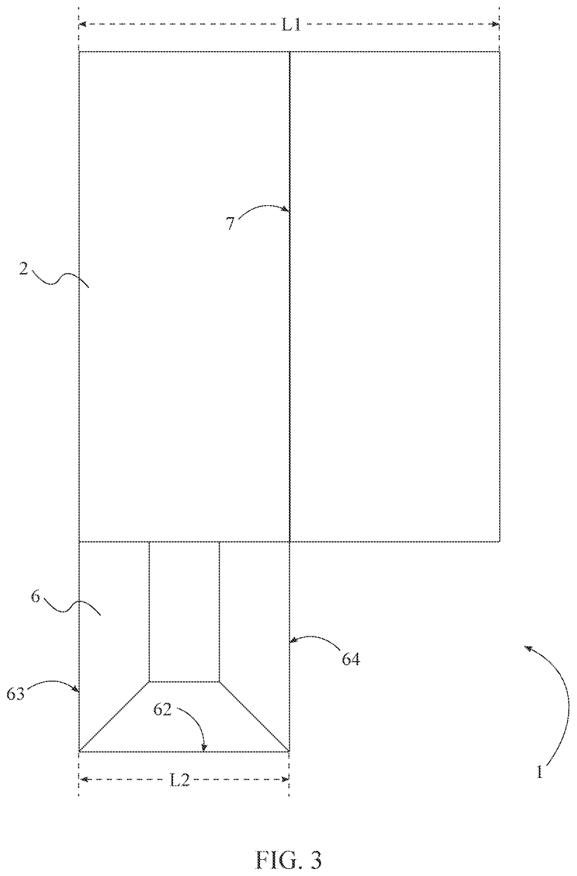

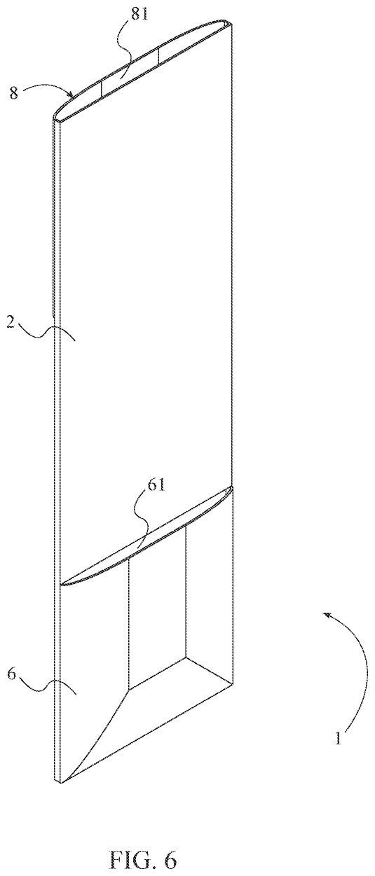

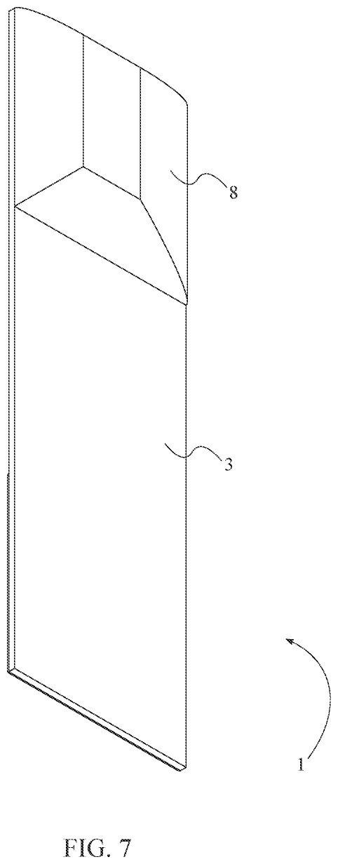

To accomplish the objectives above, the foldable bag 1 comprises a front side 2, a rear side 3, an upper section 4, and a lower section 5. The front side 2 is opposite the rear side 3 along the same sheet of plastic material. The upper section 4 is wider than the lower section 5, thereby forming a dual rectangular shape as seen in FIG. 3. More specifically, a width L1 of the upper section 4 is greater than a width L2 of the lower section 5. In the preferred embodiment, the width L1 of the upper section is twice the width L2 of the lower section. This arrangement allows the upper section 4 to effectively double in size when unfolded, thereby forming a large barrier surface for changing a baby's diaper. When folded back in place, the foldable bag 1 forms a single rectangular shape ready to be twisted, rolled and tucked, as seen in FIGS. 6 and 7.

The front side 2 is designed to receive an odorous diaper. In the preferred embodiment, the front side 2 comprises a front pocket 6 and a front folding crease 7. As best seen in FIG. 1, the front pocket 6 is positioned on the lower section 5 along the front side 2, preferably covering the entire surface of the lower section 5. The front pocket 6 is a rectilinear-shaped cavity, having a top opening flap 61 and a plurality of enclosed sides 62, 63, 64. The top opening flap 61 is designed to open up and receive a diaper or object.

As seen in FIG. 3, the front folding crease 7 traverses longitudinally along the upper section 4, preferably centered about the upper section 4 and aligned with the right side 64 of the front pocket 6. However, in other embodiments, the front folding crease 7 can be offset and not aligned with the right side 64 of the front pocket 6. The front folding crease 7 allows the front side 2 to fold in half in either direction. In an unfolded configuration, the upper section 4 on the front side 2 is designed to create a wider profile to form a barrier between the baby and the changing surface while changing the diaper.

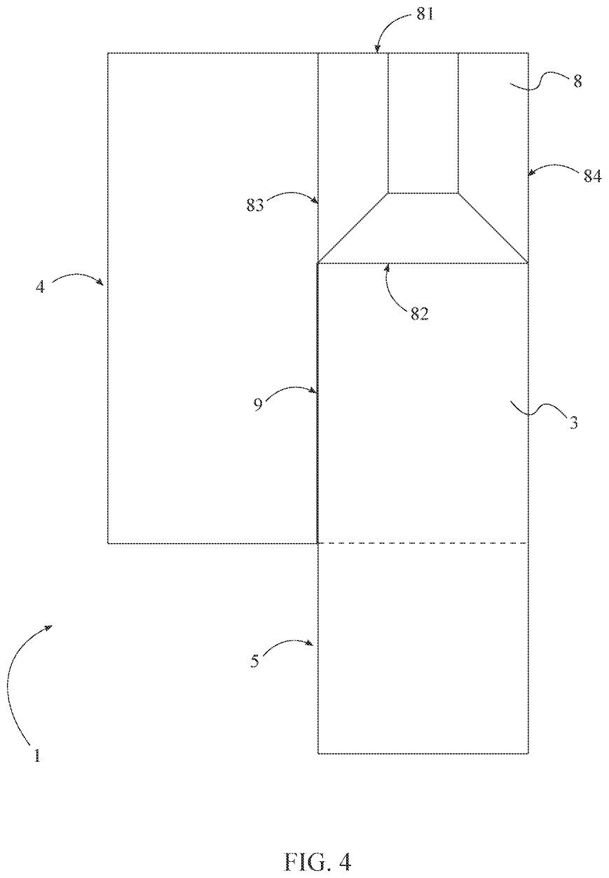

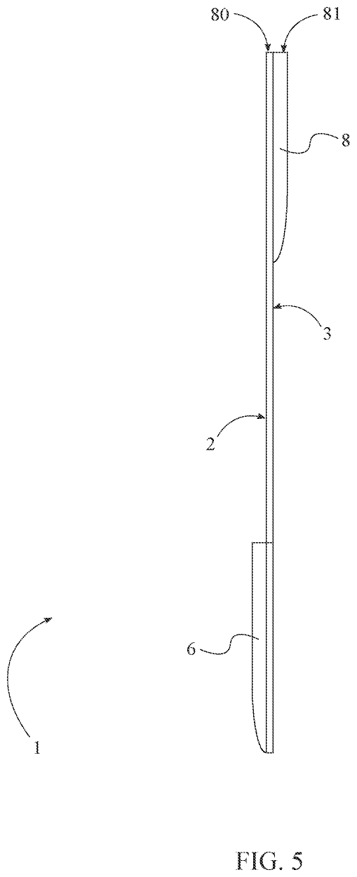

As seen in FIGS. 4-5, the rear side 3 is positioned along the opposite side of the front side 2 on the same plastic material. As such, the rear side 3 takes on the same shape as the front side 2 (i.e., dual rectangular shape). The rear side 3 comprises a rear pocket 8 and a rear folding crease 9. The rear pocket 8 is positioned on the upper section 4 along the rear side 3. The rear pocket 8 is a rectilinear-shaped cavity, having a top opening flap 81 and a plurality of enclosed sides 82, 83, 84. Preferably, the top opening flap 81 is aligned with the top edge 80 of the rear side 8. In this arrangement, the rear pocket 8 is designed to receive the front pocket 6 containing the dirty diaper or any other object.

The rear folding crease 9 traverses longitudinally about the center of the upper section 4 and is aligned with the front folding crease 7 from the opposite side. Stated another way, the rear folding crease 9 is operably coupled to the front folding crease 7, such that the upper section 4 is capable of folding in either direction. The rear folding crease 9 allows the upper section 4 on the rear side 3 to fold in half, covering the diaper in multiple layers of plastic. Preferably, the left side 83 of the rear pocket 8 is positioned adjacent to the rear folding crease 9. This arrangement ensures that the rear pocket 8 does not fold with upper section 4. However, in other embodiments, the rear folding crease 9 can be offset from and not adjacent to the left side 83 of the rear pocket 8.

It is understood that the shape of each of the front side 2 and the rear side 3 is not limited and can take the form of any suitable shape based on design, user, and/or manufacturing requirements. Similarly, the shape of each of the front pocket 6 and the rear pocket 8 is not limited and can take the form of any suitable shape based on design, user, and/or manufacturing requirements.

In an alternative embodiment, the front side 2 and the rear side 3 are designed with various colors and various sizes to accommodate larger sized adult diapers, or other odorous trash. With all the components working in tandem with each other, it is understood that the present invention is a foldable bag capable of receiving a dirty diaper, locking in germs and odor, twisting, and rolling up into a compact profile ready for disposal.

Although the invention has been explained in relation to its preferred embodiment, it is to be understood that many other possible modifications and variations can be made without departing from the spirit and scope of the invention.

What is claimed is:

1. A foldable bag for diaper disposal, the foldable bag comprising:
a front side;
a rear side;
an upper section;
a lower section;
the front side comprising a front pocket;
the front pocket being positioned on the lower section;
the front pocket having a top opening flap;
the rear side comprising a rear pocket;
the rear pocket being positioned on the upper section;
the rear pocket having a top opening flap;
the front pocket being capable of storing a used diaper; and
the front pocket capable of being rolled upward and tucked into the rear pocket.

2. The foldable bag as claimed in claim 1 comprising:
the upper section having a width greater than a width of the lower section;
the front side further comprising a front folding crease;
the front folding crease traversing longitudinally along the upper section; and
the upper section capable of folding over along the front side.

3. The foldable bag as claimed in claim 2 comprising:
the width of the upper section being twice the width of the lower section; and
the front folding crease being centrally positioned about the upper section.

4. The foldable bag as claimed in claim 3 comprising:
the front pocket being rectilinear in shape; and
a right side of the front pocket being aligned with the front folding crease.

5. The foldable bag as claimed in claim 2 comprising:
the rear side further comprising a rear folding crease;
the rear folding crease being operably coupled to the front folding crease; and
the upper section capable of folding over along the rear side.

6. The foldable bag as claimed in claim 5 comprising:
the rear pocket being rectilinear in shape; and
a left side of the rear pocket being adjacent to the rear folding crease.

7. The foldable bag as claimed in claim 6, wherein the top opening flap of the rear pocket is aligned with the top edge of the rear side.

8. The foldable bag as claimed in claim 1, wherein the foldable bag is made from a plastic material.

9. The foldable bag as claimed in claim 8, wherein the plastic material is recyclable.

10. A foldable bag for diaper disposal, the foldable bag comprising:
a front side;
a rear side;
an upper section;
a lower section;
the front side comprising a front pocket and a front folding crease;
the front pocket being positioned on the lower section;
the upper section having a width greater than a width of the lower section;
the front folding crease traversing longitudinally along the upper section;
the front pocket having a top opening flap;
the rear side comprising a rear pocket;
the rear pocket being positioned on the upper section;
the rear pocket having a top opening flap;
the foldable bag being made from a plastic material;
the upper section capable of folding over along the front side;
the front pocket being capable of storing a used diaper; and
the front pocket capable of being rolled upward and tucked into the rear pocket.

11. The foldable bag as claimed in claim 10 comprising:
the width of the upper section being twice the width of the lower section; and
the front folding crease being centrally positioned about the upper section.

12. The foldable bag as claimed in claim 11 comprising:
the front pocket being rectilinear in shape; and
a right side of the front pocket being aligned with the front folding crease.

13. The foldable bag as claimed in claim 10 comprising:
the rear side further comprising a rear folding crease;
the rear folding crease being operably coupled to the front folding crease; and
the upper section capable of folding over along the rear side.

14. The foldable bag as claimed in claim 13 comprising:
the rear pocket being rectilinear in shape; and
a left side of the rear pocket being adjacent to the rear folding crease.

15. The foldable bag as claimed in claim 14, wherein the top opening flap of the rear pocket is aligned with a top edge of the rear side.

16. The foldable bag as claimed in claim 10, wherein the plastic material is recyclable.

17. A foldable bag for diaper disposal, the foldable bag comprising:
a front side;
a rear side;
an upper section;
a lower section;
the front side comprising a front pocket and a front folding crease;
the front pocket being positioned on the lower section;
the upper section having a width twice the width of the lower section;
the front folding crease traversing longitudinally along the upper section;
the front folding crease being centrally positioned about the upper section;
the front pocket being rectilinear in shape, having a top opening flap;
a right side of the front pocket being aligned with the front folding crease;
the rear side comprising a rear pocket;
the rear pocket being positioned on the upper section;
the rear pocket having a top opening flap;
the foldable bag being made from a plastic material;
the upper section capable of folding over along the front side;
the front pocket being capable of storing a used diaper; and
the front pocket capable of being rolled upward and tucked into the rear pocket.

18. The foldable bag as claimed in claim 17 comprising:
the rear side further comprising a rear folding crease;
the rear folding crease being operably coupled to the front folding crease; and
the upper section capable of folding over along the rear side.

19. The foldable bag as claimed in claim 18 comprising:
the rear pocket being rectilinear in shape;
a left side of the rear pocket being adjacent to the rear folding crease; and
the top opening flap of the rear pocket being aligned with a top edge of the rear side.

20. The foldable bag as claimed in claim 17, wherein the plastic material is recyclable.
